(19) United States
(12) Patent Application Publication
(10) Pub. No.: US 2014/0170080 A1
(43) Pub. Date: Jun. 19, 2014

(54) ANTIOXIDANT TOPICAL COMPOSITIONS

(71) Applicant: STIEFEL RESEARCH AUSTRALIA PTY LTD., Rowville, Victoria (AU)

(72) Inventors: Lilian Fuchshuber, Rowville (AU); Robert James Houlden, Rowville (AU)

(73) Assignee: STIEFEL RESEARCH AUSTRALIA PTY LTD., Rowville, Victoria (AU)

(21) Appl. No.: 14/236,096

(22) PCT Filed: Aug. 10, 2012
(86) PCT No.: PCT/AU2012/000949

§ 371 Date: Jan. 30, 2014

Related U.S. Application Data
(60) Provisional application No. 61/522,377, filed on Aug. 11, 2011.

(51) Int. Cl. A61K 8/34; A61Q 19/08; A61Q 19/00; A61K 8/67; A61K 8/04

(52) U.S. Cl. CPC A61K 8/34; A61K 8/676; A61K 8/046; A61Q 19/00; A61Q 19/08
USPC 424/45

(57) ABSTRACT

The present invention relates to topical aerosol compositions comprising L-ascorbic acid. The compositions comprise (a) L-ascorbic acid, or a pharmaceutically acceptable salt or ester thereof, (b) water, (c) lower alcohol and (d) an aerosol propellant. In particular embodiments of the invention, the formulations are actuated as an aerosol spray or aerosol foam. The formulations are suitable for minimizing aging of the skin.

### FIELD OF THE INVENTION

The present invention relates to topical cosmetic compositions comprising L-ascorbic acid.

### BACKGROUND OF THE INVENTION

Free radicals are known to damage cells including skin cells. It is thought that the use of antioxidants to scavenge free radicals can minimise such damage. One such antioxidant, L-ascorbic acid (also known as vitamin C), has the following structure:

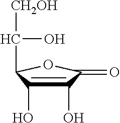

L-Ascorbic acid has been marketed in various topical preparations for anti-aging, skin rejuvenation and skin-lightening purposes. L-Ascorbic acid is also thought to minimise UV damage. Exemplary topical L-Ascorbic acid preparations available on the market include SkinCeuticals™ serum (SkinCeuticals), CelIecC™ serum/cream (Cellec-C International, Inc.) and C'ensil™ serum (Grand Aespio, Inc.).

L-Ascorbic acid is highly unstable and thus presents a significant challenge to the formulation chemist. In particular, L-Ascorbic acid is unstable in aqueous solution and is sensitive to air, light and elevated temperatures. Accordingly, there remains a need in the art for highly stable topical L-Ascorbic acid preparations in alternative topical dosage forms comprising this vitamin, such as an aerosol spray or foam.

### SUMMARY OF THE INVENTION

In one embodiment, the present invention provides a topical cosmetic composition in an aerosol container comprising (a) L-ascorbic acid or a pharmaceutically acceptable salt or ester thereof, (b) water, (c) a lower alcohol and (d) an aerosol propellant.

According to another embodiment, the invention provides a method of treating a condition of the skin in a mammal in need thereof, the method comprising administering to said mammal a topical cosmetic composition in an aerosol container comprising (a) L-ascorbic acid or a pharmaceutically acceptable salt or ester thereof, (b) water, (c) a lower alcohol and (d) an aerosol propellant.

According to a further embodiment, the invention relates to the use of a topical cosmetic composition in an aerosol container comprising (a) L-ascorbic acid or a pharmaceutically acceptable salt or ester thereof, (b) water, (c) a lower alcohol and (d) an aerosol propellant, in the manufacture of a cosmetic for the treatment of a condition of the skin.

In yet a further embodiment, the invention relates to the use of a cosmetic composition in an aerosol container comprising (a) L-ascorbic acid or a pharmaceutically acceptable salt or ester thereof, (b) water, (c) a lower alcohol and (d) an aerosol propellant, for the treatment of a condition of the skin.

### BRIEF DESCRIPTIONS OF THE DRAWINGS

FIG. 1 illustrates the stability of aerosol spray formulations containing L-ascorbic acid, ascorbyl tetraisopalmitate or ascorbic acid 6-palmitate.

FIG. 2 illustrates the stability of further aerosol spray formulations containing L-ascorbic acid.

FIG. 3 illustrates the stability of aerosol spray formulations containing L-ascorbic acid utilising various alternative aerosol propellants.

FIG. 4 illustrates the stability of aerosol foams containing L-ascorbic acid.

FIG. 5 illustrates the stability of aerosol foams containing L-ascorbic acid.

FIG. 6 illustrates the stability of aerosol foams containing L-ascorbic acid and Coffeeberry™ extract.

FIG. 7 illustrates the stability of aerosol foams containing L-ascorbic acid and Coffeeberry™ extract.

FIG. 8 illustrates the stability of aerosol foams containing L-ascorbic acid and Coffeeberry™ extract.

### DETAILED DESCRIPTION OF THE INVENTION

The present invention provides a topical cosmetic composition in an aerosol container comprising (a) L-ascorbic acid or a pharmaceutically acceptable salt or ester thereof, (b) water, (c) a lower alcohol and (d) an aerosol propellant.

According to an embodiment, the topical cosmetic composition forms an aerosol spray or an aerosol foam after release from the aerosol container. In one embodiment, the topical cosmetic composition forms an aerosol spray after release from the aerosol container. In an alternative embodiment, the topical cosmetic composition forms an aerosol foam after release from the aerosol container.

In one particular embodiment, the L-ascorbic acid or pharmaceutically acceptable salt or ester thereof is selected from the group consisting of L-ascorbic acid, sodium L-ascorbate, calcium L-ascorbate, ascorbyl tetraisopalmitate and ascorbic acid 6-palmitate, and mixtures thereof. In one embodiment, the formulation comprises L-ascorbic acid. In another embodiment, the formulation comprises a mixture of L-ascorbic acid and a pharmaceutically acceptable salt or ester of L-ascorbic acid. The salt or ester of L-ascorbic acid may be a solvate or hydrate as is well known and defined in the art. In another embodiment, the formulation comprises L-ascorbic acid, and an L-ascorbic acid salt or ester which is ascorbyl tetraisopalmitate or ascorbic acid 6-palmitate, or a combination thereof.

The L-ascorbic acid, salt or ester thereof is suitably present in an amount from about 1% to about 20% by weight, based on the total weight of the composition. In another embodiment, the L-ascorbic acid, salt or ester thereof is present in an amount from about 2% to about 15% by weight, based on the total weight of the composition. In one particular embodiment, the L-ascorbic acid or pharmaceutically acceptable salt or ester thereof is present in an amount of about 5% by weight. In another embodiment, the L-ascorbic acid or pharmaceutically acceptable salt or ester thereof is present in an amount of about 12% by weight.

The present topical compositions comprise water. Suitably, the water is present in an amount from about 10% to about 80% by weight. In one embodiment, the water is present in an amount from about 20% to about 60% by weight. In another embodiment, the water is present in an amount from about 40% to about 60% by weight. In yet another embodiment, the water is present in an amount from about 15% to about 45% by weight. In a further embodiment, the composition comprises water in an amount from about 15% to about 30% by weight. In yet a further embodiment, the composition comprises water in an amount from about 25% to about 45% by weight.

The cosmetic composition also comprises a lower alcohol, such as a (C₁-C₆) alcohol. The alkyl moiety of the alcohol represents a branched or straight chain hydrocarbon moiety, such as methyl, ethyl, n-propyl, isopropyl, n-butyl, s-butyl, isobutyl, t-butyl and the like. When substituted by an alcohol, exemplary lower alcohols can include, but are not limited to, ethanol, propanol, isopropanol, n-butyl alcohol and t-butyl alcohol, and mixtures thereof. In one embodiment, the lower alcohol is a single alcohol, such as ethanol. In another embodiment, the lower alcohol is a mixture of alcohols, such as ethanol and at least one other lower alcohol.

Suitably, the lower alcohol is present in the composition in an amount from about 10% to about 80% by weight. In one embodiment, the lower alcohol is present in an amount from about 20% to about 60% by weight. In another embodiment, the lower alcohol is present in an amount from about 20% to about 40% by weight. In another embodiment, the lower alcohol is present in an amount from about 35% to about 65% by weight. In yet another embodiment, the lower alcohol is present in an amount from about 35% to about 50% by weight. In a further embodiment, the lower alcohol is present in an amount from about 40% to about 65% by weight. In one embodiment, the lower alcohol is ethanol present in these amounts. In another embodiment, the lower alcohol is a mixture of alcohols, such as ethanol and at least one other lower alcohol present in these amounts.

The cosmetic composition comprises an aerosol propellant. In an embodiment, the aerosol propellant is selected from the group consisting of a hydrocarbon, dimethyl ether, a chlorofluorocarbon, a hydrofluorocarbon, and mixtures thereof. Other suitable propellants include compressed gases such as nitrogen, carbon dioxide, nitrous oxide and air.

In one embodiment, the aerosol propellant is a hydrocarbon propellant. Suitably, the hydrocarbon propellant is a mixture of hydrocarbons. In one embodiment, the hydrocarbon is selected from the group consisting of propane, n-butane and isobutane, and mixtures thereof. In another embodiment, the aerosol propellant is dimethyl ether. In yet another embodiment, the aerosol propellant is a hydroflurocarbon propellant, such as 1,1,1,2-tetrafluroethane (hydrofluorocarbon 134a). One of skill in the art would appreciate that other combinations of propellant are useful in the present invention.

Suitably, the aerosol propellant is present in an amount from about 1% to about 50% by weight. In one embodiment, the aerosol propellant is present in an amount from about 25% to about 40% by weight, such as about 30% to about 35% by weight. In an alternative embodiment, the aerosol propellant is present in an amount from about 1% to about 20% by weight, such as from about 3% to about 15% by weight or from about 3% to about 10% by weight.

### Dermatologically Acceptable Excipients

The present topical cosmetic compositions may comprise one or more dermatologically acceptable excipients.

Suitably, the dermatologically acceptable excipient is selected from the group consisting of a preservative, a second antioxidant, a chelating agent, a humectant, a pH adjusting agent, a wax, a surfactant, and mixtures thereof.

In one embodiment, the dermatologically acceptable excipient is selected from the group consisting of a preservative, a second antioxidant, a chelating agent, a humectant and a pH adjusting agent, and mixtures thereof.

Other suitable excipients include fragrances, colorants, skin conditioning agents and penetration enhancers.

These dermatologically acceptable excipients will be discussed in more detail below.

### Preservatives

The present topical cosmetic compositions may comprise a preservative. In an embodiment, the preservative is a mixture of two or more preservatives.

Exemplary preservatives include, but are not limited to, benzyl alcohol, diazolidinyl urea, methyl paraben, ethyl paraben, propyl paraben, butyl paraben, phenoxyethanol, sorbic acid, benzoic acid, salts thereof, or a combination or mixture thereof.

In one embodiment, the preservative is benzyl alcohol. In another embodiment, the preservative is phenoxyethanol.

Suitably, the preservative is present in the composition in an amount from about 0.01% to about 2% by weight.

### Second Antioxidant

The present topical cosmetic compositions may comprise a second antioxidant (i.e. in addition to the L-ascorbic acid). In one embodiment, the function of the second antioxidant is to act as a sacrificial antioxidant and thus maximise the stability of the L-ascorbic acid, salt or ester thereof in the composition. In an embodiment, the second antioxidant is a mixture of two or more antioxidants.

Exemplary second antioxidants include, but are not limited to, butylated hydroxytoluene (BHT), butylated hydroxyanisole, tocopherol, propyl gallate, vitamin E TPGS, or a combination or mixture thereof.

In one embodiment, the second antioxidiant is BHT. In another embodiment, the second antioxidiant is propyl gallate. In yet another embodiment, the second antioxidant is mixture of BHT and propyl gallate. In a further embodiment, the second antioxidant is tocopherol. Suitably, the second antioxidant is present in the composition in an amount from about 0.001% to about 1% by weight.

### Chelating Agents

The present topical cosmetic compositions may comprise a chelating agent. In an embodiment, the chelating agent is a mixture of two or more chelating agents.

Exemplary chelating agents include, but are not limited to, citric acid, glucuronic acid, sodium hexametaphosphate, zinc hexametaphosphate, ethylene diamine tetraacetic acid (EDTA), phosponates, salts thereof, or a combination or mixture thereof.

In one embodiment, the chelating agent is EDTA. In an alternative embodiment, the chelating agent is citric acid.

Suitably, the chelating agent is present in the composition in an amount from about 0.1% to about 1% by weight.

### Humectant

The topical cosmetic compositions may comprise a humectant. In an embodiment, the humectant is a mixture of two or more humectants.

Exemplary humectants include, but are not limited to, glycerol, sorbitol, maltitol, polydextrose, triacetin, propylene glycol, and polyethylene glycols (PEG) such as PEG-4, PEG-6, PEG-8, PEG-12, PEG-32, PEG-75 and PEG-150, or a combination or mixture thereof.

In an embodiment, the humectant is glycerol. In another embodiment, the humectant is propylene glycol. In yet another embodiment, the humectant is a polyethylene glycol.

Suitably, the humectant is present in the composition in an amount from about 0.1% to about 40% by weight, such as about 0.2% to about 20% by weight. In one embodiment, the humectant is present in the composition in an amount from about 0.5% to about 5% by weight.

### pH Adjusting Agent

The present topical cosmetic compositions may comprise a pH adjusting agent. In one embodiment, the pH adjusting agent is a base or mixture thereof. Suitable bases include amines, bicarbonates, carbonates, and hydroxides such as alkali or alkaline earth metal hydroxides, as well as transition metal hydroxides. In an embodiment, the base is sodium hydroxide or potassium hydroxide.

In another embodiment, the pH adjusting agent is an acid, an acid salt, or mixtures thereof. Suitably, the acid is selected from the group consisting of lactic acid, acetic acid, maleic acid, succinic acid, citric acid, phosphoric acid, nitric acid, sulphuric acid and hydrochloric acid, or a combination or mixture thereof.

In yet another embodiment, the pH adjusting agent is a buffer. Suitably, the buffer is selected from the group consisting of citrate/citric acid, acetate/acetic acid, phosphate/phosphoric acid, propionate/propionic acid, lactate/lactic acid, carbonate/carbonic acid, ammonium/ammonia and edetate/edetic acid, or a combination or mixture thereof. Suitably, the buffer is citrate/citric acid. The function of the buffer is to set or maintain the pH of the composition at a constant, or near constant value.

Suitably, the pH adjusting agent is present in the composition in an amount from about 0.01% to about 10% by weight. In an alternative embodiment, the pH adjusting agent is present in an amount sufficient to adjust or maintain the pH of the composition in the container between about pH 2 to about pH 6, such as between about pH 4 to about pH 6.

### Wax

The present topical cosmetic compositions may comprise a wax. In an embodiment, the wax is a mixture of two or more waxes.

Exemplary waxes include, but are not limited to, fatty alcohols, beeswax and derivatives thereof such as PEG-8 beeswax, PEG-12 beeswax and PEG-20 beeswax, jojoba wax, lanolin wax, rice wax, cholesterol, and combinations or mixtures thereof.

Suitably, the wax is present in an amount from about 0.1% to about 5% by weight.

In one embodiment, the wax is a fatty alcohol. In another embodiment, the wax is a mixture of two or more fatty alcohols.

Exemplary fatty alcohols include, but are not limited to, caprylic alcohol, decyl alcohol, lauryl alcohol, myristyl alcohol, behenyl alcohol, lanolin alcohol, arachidyl alcohol, oleyl alcohol, palm alcohol, isocetyl alcohol, cetyl alcohol and stearyl alcohol, or a combination or mixture thereof.

In one embodiment, the fatty alcohol is stearyl alcohol. In another embodiment, the fatty alcohol is cetyl alcohol. In yet another embodiment, the fatty alcohol is a mixture of stearyl alcohol and cetyl alcohol. Suitably, the ratio of stearyl alcohol to cetyl alcohol is about 1:1 to about 1:3.

Suitably, when the wax is a fatty alcohol it is present in the composition in an amount from about 0.1% to about 5% by weight. In one embodiment, the fatty alcohol is present in the composition in an amount from about 0.5% to about 4% by weight.

### Surfactant

The present topical cosmetic compositions may comprise at least one surfactant. In an embodiment, the surfactant is a mixture of two or more surfactants.

In one embodiment, the surfactant consists of one or more non-ionic surfactants. Suitable non-ionic surfactants include, but are not limited to, ethoxylated fatty alcohol ethers, PEG derivatives, ethoxylated fatty acids, propylene glycol esters, glycerol esters and derivatives, polymeric ethers and sorbitan derivatives, and mixtures thereof.

In one embodiment, the surfactant is an ethoxylated fatty alcohol ether. Exemplary ethoxylated fatty alcohol ethers include ceteth-1, ceteth-2, ceteth-3, ceteth-4, ceteth-5, ceteth-6, ceteth-10, ceteth-12, ceteth-14, ceteth-15, ceteth-16, ceteth-20, ceteth-24, ceteth-25, ceteth-30, ceteth-45, steareth-2, steareth-10, steareth-20, ceteareth-2, ceteareth-3, ceteareth-5, ceteareth-6, ceteareth-10, ceteareth-12, ceteareth-15, ceteareth-20, ceteareth-21, ceteareth-22, ceteareth-25, ceteareth-30, ceteareth-31, ceteareth-32, ceteareth-33, laureth-3, laureth-4, laureth-5, laureth-9, laureth-10, laureth-12, laureth-15, laureth-20, laureth-21, laureth-22, laureth-23, nonoxynol-9, oleth-2, oleth-5, oleth-10 and oleth-20, and mixtures thereof.

In another embodiment, the surfactant is a hydrophilic ethoxylated fatty alcohol ether. Exemplary hydrophilic ethoxylated fatty alcohol ethers include ceteth-6, ceteth-10, ceteth-12, ceteth-14, ceteth-15, ceteth-16, ceteth-20, ceteth-24, ceteth-25, ceteth-30, ceteth-45, steareth-10, steareth-20, ceteareth-10, ceteareth-12, ceteareth-15, ceteareth-20, ceteareth-21, ceteareth-22, ceteareth-25, ceteareth-30, ceteareth-31, ceteareth-32, ceteareth-33, ceteareth-6, laureth-5, laureth-9, laureth-10, laureth-12, laureth-15, laureth-20, laureth-21, laureth-22, laureth-23, nonoxynol-9, oleth-10 and oleth-20, and mixtures thereof.

In one embodiment, the ethoxylated fatty alcohol ether is steareth-20.

In yet another embodiment, the surfactant is a PEG derivative. Exemplary PEG derivatives include PEG-7 hydrogenated castor oil, PEG-25 hydrogenated castor oil, PEG-30 castor oil, PEG-31 castor oil, PEG-32 castor oil, PEG-33 castor oil, PEG-34 castor oil, PEG-35 castor oil, PEG-40 hydrogenated castor oil, PEG-50 castor oil and PEG-60 hydrogenated castor oil, and mixtures thereof.

In a further embodiment, the surfactant is an ethoxylated fatty acid. Exemplary ethoxylated fatty acids include PEG-5 oleate, PEG-6 oleate, PEG-10 oleate, PEG-6 stearate, PEG-8 stearate, PEG-9 stearate, PEG-20 stearate, PEG-40 stearate, PEG-41 stearate, PEG-42 stearate, PEG-43 stearate, PEG-44 stearate, PEG-45 stearate, PEG-46 stearate, PEG-47 stearate, PEG-48 stearate, PEG-49 stearate, PEG-50 stearate and PEG-100 stearate, and mixtures thereof.

In yet a further embodiment, the surfactant is a propylene glycol ester. Exemplary propylene glycol esters include propylene glycol palmitate and propylene glycol stearate, and mixtures thereof.

In one embodiment, the surfactant is a glyceryl ester or derivative thereof. A derivative can include ethoxylated glyceryl esters or a polyglyceryl esters. Exemplary glyceryl esters and derivatives include glyceryl behenate, glyceryl dibehenate, glyceryl dioleate, glyceryl distearate, glyceryl linoleate, glyceryl oleate, glyceryl stearate, PEG-23 glyceryl cocoate, PEG-6 caprylic/capric glycerides, PEG-7 glyceryl cocoate, polyglyceryl-diisostearate, polyglyceryl-2 diisostearate, polyglyceryl-3 diisostearate and polyglyceryl-6 diisostearate, and mixtures thereof.

In another embodiment, the surfactant is a polymeric ether. Exemplary polymeric ethers include poloxamer 124, poloxamer 182, poloxamer 184, poloxamer 188, poloxamer 237, poloxamer 331, poloxamer 338 and poloxamer 407, and mixtures thereof.

In another embodiment, the surfactant is a sorbitan derivative. Exemplary sorbitan derivatives include polysorbate 20, polysorbate 40, polysorbate 60, polysorbate 80, sorbitan laurate, sorbitan oleate, sorbitan palmitate, sorbitan sesquioleate, sorbitan stearate and sorbitan trioleate, and mixtures thereof. In one embodiment, the sorbitan derivative is polysorbate 60.

Suitably, the at least one surfactant is present in the composition in an amount from about 0.1% to about 15% by weight. In one embodiment, the surfactant is present in an amount from about 0.1% to about 5% by weight.

### Nutritional Agent

According to an embodiment, the topical cosmetic composition may further comprise a nutritional agent. In an embodiment, the nutritional agent is a mixture of two or more nutritional agents. The purpose of the nutritional agent is to act in concert with the L-ascorbic acid, salt or ester thereof to improve the cosmetic effect of the composition.

Exemplary nutritional agents include vitamins, coenzymes, fruit extracts, plant extracts, and mixtures thereof.

In an embodiment, the nutritional agent is a vitamin. Exemplary vitamins include vitamins A, B, D, E and K. In one embodiment, the vitamin is vitamin A. In another embodiment, the vitamin is vitamin E. In another embodiment, the vitamin is a mixture of vitamin A and vitamin E.

In another embodiment, the nutritional agent is a coenzyme. In one embodiment, the coenzyme is ubiquinone (coenzyme Q10).

In yet another embodiment, the nutritional agent is a fruit extract. Exemplary fruit extracts include, but are not limited to, Arbutus Unedo fruit extract, Arctium Lappa extract, Bacillus/Hippophae Rhamnoides fruit ferment extract, Bergamot/Grapefruit/Orange/Tangerine fruit ferment extract, Capparis Moonii fruit extract, Capsicum Annuum fruit extract, Chaenomeles Sinensis fruit extract, Citrus Medica Vulgaris fruit extract, Citrus Unshiu fruit extract, Coffea Arabica fruit extract, Davidsonia Pruriens fruit extract, Empetrum Nigrum Flower/Fruit/Leaf extract, Forsythia Suspensa fruit extract, Grifola Frondosa Fruiting Body extract, Hydrolyzed Annona Cherimolia fruit extract, Hydrolyzed Lycium Barbarum fruit extract, Hydrolyzed Olive fruit extract, Hydrolyzed Sophora Japonica fruit extract, Lactobacillus/Lycium Chinense fruit extract ferment filtrate, Lactobacillus/Saccharomyces/Euterpe Oleracea fruit extract ferment filtrate, Lactobacillus/Scutellaria Baicalensis Root/Camellia Sinensis Leaf/Artemisia Princeps Leaf/Houttuynia Cordata Leaf/Citrus Junos fruit extract ferment filtrate, Lycium Chinense fruit extract, Malpighia Punicifolia (Acerola) fruit extract, Malus Domestica fruit extract, Momordica Grosvenorii fruit extract, Morus Alba fruit extract, Musa Balbisiana fruit extract, Prunus Cerasus (Bitter Cherry) fruit extract, Prunus Salicina fruit extract, Prunus Tomentosa fruit extract, Psidium Cattleianum fruit extract, Punica Granatum Bark/fruit extract, Quercus Infectoria fruit extract, Rubus Arcticus fruit extract, Rubus Chamaemorus fruit extract, Santalum Acuminatum fruit extract, Sapindus Oahuensis fruit extract, Schizandra Sphenanthera fruit extract, Smilax China fruit extract, Solanum Lycopersicum (Tomato) fruit extract, Syzygium Leuhmanii fruit extract, Terminalia Ferdinandiana fruit extract, Vaccinium Vitis-Idaea fruit extract, and Vitis Vinifera (Grape) fruit extract, and mixtures thereof. In one embodiment, the fruit extract is Coffea Arabica fruit extract.

In a further embodiment, the nutritional agent is a plant extract. Exemplary plant extracts include Physalis Angulata extract, Piptadenia Colubrina Peel extract, Grape (Vitis Vinifera) leaf extract, Camellia Oleifera seed extract, Camellia Oleifera leaf extract and Camellia sinensis leaf extract, and mixtures thereof. In one embodiment, the plant extract is Camellia sinensis leaf extract, also known as Green tea extract. In another embodiment, the plant extract is Piptadenia Colubrina Peel extract. In yet another embodiment, the plant extract is Physalis Angulata extract. In a further embodiment, plant extract is a mixture of Piptadenia Colubrina Peel extract and Physalis Angulata extract.

Suitably, the nutritional agent is present in the composition in an amount from about 0.1% to about 20% by weight, depending on the nature of the nutritional agent. In one embodiment, the nutritional agent is present in the composition in an amount from about 0.1% to about 5% by weight. In another embodiment, the nutritional agent is present in the composition in an amount from about 0.5% to about 2% by weight.

### The Container

The topical cosmetic composition is packaged in an aerosol container. The container is selected to provide the composition with a long shelf life and, accordingly, should be chemically inert so as to not interfere with the chemical stability of the composition.

Suitable containers may be made of, for example, steel, aluminium or glass and may employ one or more internal and/or external protective linings. In an embodiment, the aerosol container is a polyamide-imide (PAM) lined aluminium aerosol container. In another embodiment, the aerosol container is a bag in can or bag on valve assembly.

### Aerosol Spray

In one particular embodiment, the topical cosmetic composition is formulated as an aerosol spray. In this embodiment, the invention provides a topical cosmetic composition in an aerosol container comprising (a) L-ascorbic acid or a pharmaceutically acceptable salt or ester thereof, (b) water, (c) a lower alcohol and (d) an aerosol propellant, wherein the composition forms an aerosol spray after release from said container.

In an embodiment, the ratio of water to lower alcohol is from about 1:1.5 to 1:3. In another embodiment, the ratio of water to lower alcohol is from about 1:1.75 to 1:2.5. In yet another embodiment, the ratio of water to lower alcohol is from about 1:2 to about 1:2.2. Suitably, the ratio of water to lower alcohol is about 1:2, 1:2.05, 1:2.1, 1:2.15 or 1:2.2.

In one embodiment, the amount of water present in the aerosol spray is from about 15% to about 30% by weight. In one embodiment, the amount of lower alcohol present in the aerosol spray is from about 35% to about 50% by weight. In one embodiment, the amount of propellant present in the aerosol spray is from about 25% to about 40% by weight.

Accordingly, in one embodiment, the invention provides a topical cosmetic composition in an aerosol container comprising (a) L-ascorbic acid or a pharmaceutically acceptable salt or ester thereof, (b) water in an amount from about 15% to about 30% by weight, (c) a lower alcohol in an amount from about 35% to about 50% by weight and (d) an aerosol propellant in an amount from about 25% to about 40% by weight, wherein the composition forms an aerosol spray after release from said container. Optionally this formulation may further comprise one or more dermatologically acceptable excipients selected from the group consisting of a preservative, a second antioxidant, a chelating agent and a pH adjusting agent.

In another embodiment, the invention provides a topical cosmetic composition in an aerosol container comprising (a) L-ascorbic acid or a pharmaceutically acceptable salt or ester thereof, (b) water in an amount from about 15% to about 30% by weight, (c) a lower alcohol in an amount from about 35% to about 50% by weight, (d) one or more dermatologically acceptable excipients selected from the group consisting of a preservative, a second antioxidant, a chelating agent and a pH adjusting agent, (e) an aerosol propellant in an amount from about 25% to about 40% by weight, wherein the composition forms an aerosol spray after release from said container.

### Aerosol Foam

In the alternative, the composition may be formulated as an aerosol foam. In these embodiments, the composition comprises in an aerosol container (a) L-ascorbic acid or a pharmaceutically acceptable salt or ester thereof, (b) water, (c) a lower alcohol and (d) an aerosol propellant, wherein the composition forms an aerosol foam after release from said container.

In an embodiment, less than 10% of the L-Ascorbic acid or salt or ester thereof is lost after 30 weeks of storage of the aerosol foam, composition at 25° C.

Accordingly, in one embodiment, the water present in the aerosol foam is in an amount from about 20% to about 60% by weight. In one embodiment, the lower alcohol present in the aerosol form is in an amount from about 20% to about 60% by weight. In one embodiment, the aerosol propellant is present in an amount from about 3% to about 10% by weight.

In one embodiment, the aerosol foam may be formulated as a high alcohol content foam. Accordingly, in this embodiment, the ratio of water to lower alcohol is about 1:1 to about 1:2. In another embodiment, the ratio of water to lower alcohol is about 1:1.25 to about 1:1.75. In yet another embodiment, the ratio of water to lower alcohol is about 1:1.5.

In one embodiment of the high alcohol content foam, the water is present in the aerosol foam in an amount from about 25% to about 45% by weight. In another embodiment, the lower alcohol is present in an amount from about 40% to about 65% by weight. In yet another embodiment, the aerosol propellant is present in an amount from about 3% to about 10% by weight.

Accordingly, in one embodiment, the invention provides a topical cosmetic composition in an aerosol container comprising (a) L-ascorbic acid or a pharmaceutically acceptable salt or ester thereof, (b) water in an amount from about 25% to about 45% by weight, (c) a lower alcohol in an amount from about 40% to about 65% by weight, (d) an aerosol propellant in an amount from about 3% to about 10% by weight, wherein the composition forms an aerosol foam after release from said container.

In an embodiment, the aerosol foam may further comprise a wax. In another embodiment, the aerosol foam further comprises a surfactant. In yet another embodiment, the aerosol foam further comprises a wax and/or a surfactant. In a further embodiment, the aerosol foam further comprises a wax and a surfactant.

The aerosol foam may comprise one or more additional dermatologically acceptable excipients, as described. In one embodiment the aerosol foam optionally includes one or more dermatologically acceptable excipients selected from the group consisting of a preservative, a second antioxidant, a chelating agent, a humectant and a pH adjusting agent.

Accordingly, in one embodiment, the invention provides a topical cosmetic composition in an aerosol container comprising (a) L-ascorbic acid or a pharmaceutically acceptable salt or ester thereof, (b) water, (c) a lower alcohol (d) a wax, (e) a surfactant, (1) optionally one or more dermatologically acceptable excipients selected from the group consisting of a preservative, a second anti-oxidant, a chelating agent, a humectant and a pH adjusting agent, and (g) an aerosol propellant, wherein the composition forms an aerosol foam after release from said container.

In an embodiment, the aerosol foam is a quick breaking temperature sensitive foam. That is, after topical application of the foam, the foam readily melts on the skin of the user with gentle rubbing or spreading, leaving little residue on the surface of the skin.

In one embodiment, the invention provides a topical cosmetic composition in an aerosol container comprising (a) L-ascorbic acid or a pharmaceutically acceptable salt or ester thereof, (b) water in an amount from about 25% to about 45% by weight, (c) a lower alcohol in an amount from about 40% to about 65% by weight, (d) a wax, (e) a surfactant, (f) optionally one or more dermatologically acceptable excipients selected from the group consisting of a preservative, a second anti-oxidant, a chelating agent, a humectant and a pH adjusting agent, and (g) an aerosol propellant in an amount from about 3% to about 10% by weight, wherein the composition forms an aerosol foam after release from said container.

In another embodiment, the aerosol foam is formulated as a lower alcohol content foam. In this embodiment, the ratio of water to lower alcohol present in the composition is about 1:1 to about 1:0.4. In another embodiment, the ratio of water to lower alcohol is about 1:0.7 to about 1:0.5. In yet another embodiment, the ratio of water to lower alcohol is about 1:0.6.

In one embodiment of the low alcohol content foam, the water is present in the aerosol foam in an amount from about 40% to about 60% by weight. In another embodiment, the lower alcohol is present in an amount from about 20% to about 40% by weight. In yet another embodiment, the aerosol propellant is present in an amount from about 3% to about 10% by weight.

In a particular embodiment, the invention provides a topical cosmetic composition in an aerosol container comprising (a) L-ascorbic acid or a pharmaceutically acceptable salt or ester thereof, (b) water in an amount from about 40% to about 60% by weight, (c) a lower alcohol in an amount from about 20% to about 40% by weight, (d) an aerosol propellant in an amount from about 3% to about 10% by weight, wherein the composition forms an aerosol foam after release from said container.

In one particular embodiment, the invention provides a topical cosmetic composition in an aerosol container comprising (a) L-ascorbic acid or a pharmaceutically acceptable salt or ester thereof, (b) water in an amount from about 40% to about 60% by weight, (c) a lower alcohol in an amount from about 20% to about 40% by weight, (d) a wax, (e) a surfactant, (f) optionally one or more dermatologically acceptable excipients selected from the group consisting of a preservative, a second anti-oxidant, a chelating agent, a humectant and a pH adjusting agent, and (g) an aerosol propellant in an amount from about 3% to about 10% by weight, wherein the composition forms an aerosol foam after release from said container.

In one further embodiment, the invention provides a topical cosmetic composition in an aerosol container comprising (a) L-ascorbic acid or a pharmaceutically acceptable salt or ester thereof, (b) water in an amount from about 40% to about 60% by weight, (c) a lower alcohol in an amount from about 20% to about 40% by weight, (d) a wax, (e) a surfactant, (f) Coffea Arabica fruit extract, (g) optionally one or more dermatologically acceptable excipients selected from the group consisting of a preservative, a second anti-oxidant, a chelating agent, a humectant and a pH adjusting agent, and (h) an aerosol propellant in an amount from about 3% to about 10% by weight, wherein the composition forms an aerosol foam after release from said conatiner.

### Methods of Treatment

According to an embodiment, the invention provides a method of treating a condition of the skin in a mammal in need thereof, the method comprising administering to said mammal a topical cosmetic composition in an aerosol container comprising (a) L-ascorbic acid or a pharmaceutically acceptable salt or ester thereof, (b) water, (c) a lower alcohol and (d) an aerosol propellant. In an embodiment, the mammal is a human.

Suitably, the condition is aging of the skin. This may be characterised by fine lines, wrinkles, UV damage and/or age spots.

In one embodiment, the cosmetic compositions of the invention are suitable for once-daily application to the skin. In another embodiment, the compositions are used in conjunction with at least a second topical preparation, forming a beauty regimen. In particular, the present compositions may be used in conjunction with facial scrubs, washes, cleansers and toners, for example.

### Definitions

The term “administering” is used herein to mean any method which in sound cosmetic practice topically delivers the composition to a subject in such a manner as to provide the desired cosmetic effect.

The terms “treating” or “treatment” of a cosmetic condition encompasses alleviation of at least one symptom thereof, a reduction in the severity thereof, or the delay, prevention or inhibition of the progression thereof. Treatment need not mean that the condition is totally cured. A useful cosmetic composition herein need only to reduce the severity of the condition, reduce the severity of symptoms associated therewith, provide improvement to a patient's quality of life, or delay, prevent or inhibit the onset of the condition.

The term “salt” refers to salts that are cosmetically or pharmaceutically acceptable. Such salts include (1) acid addition salts, formed with acids such as, for example, acetic acid, benzoic acid, citric acid, gluconic acid, glutamic acid, glutaric acid, glycolic acid, hydrochloric acid, lactic acid, maleic acid, malic acid, malonic acid, mandelic acid, phosphoric acid, propionic acid, sorbic acid, succinic acid, sulfuric acid, tartaric acid, naturally and synthetically derived amino acids, and mixtures thereof and (2) salts formed when an acidic proton present in the parent compound is either (i) replaced by a metal ion (e.g. alkali metal ion, alkaline earth metal ion or aluminium ion) or (ii) protonates an organic base such as, for example, ethanolamine, diethanolamine, triethanolamine, tromethamine and N-methylglucamine.

Any percentage range or ratio range recited herein is to be understood to include percentages or ratios of any integer within that range and fractions thereof, such as one tenth and one hundredth of an integer, unless otherwise indicated.

It should be understood that the terms “a” and “an” as used herein refer to “one or more” of the enumerated components. It will be clear to one of ordinary skill in the art that the use of the singular includes the plural unless specifically stated otherwise.

Throughout the application, descriptions of various embodiments use “comprising” language, however in some specific instances, an embodiment can alternatively be described using the language “consisting essentially of or “consisting of”.

All numbers expressing quantities, percentages or proportions, and other numerical values used in the specification and claims, are to be understood as being modified in all instances by the term “about”.

All percentages are based on the percent by weight of the final composition prepared, and unless otherwise indicated, all totals equal 100% by weight.

Other terms used herein are intended to be defined by their well known meanings in the art.

### EXAMPLES

### Example 1

### L-Ascorbic Acid Aerosol Spray

The following L-ascorbic acid aerosol sprays were prepared:

TABLE 1

| | | Formulation Number | | | | | |
|---|---|---|---|---|---|---|---|
| | | #1 | #2 | #3 | #4 | #5 | #6 |
| Item | Ingredients | % w/w | % w/w | % w/w | % w/w | % w/w | % w/w |
| 1 | Ascorbyl tetraisopalmitate | 5.00 | | | 3.12 | | |
| 2 | Ascorbic acid 6-palmitate | | 3.00 | | | 2.00 | |
| 3 | L-Ascorbic acid | | | 5.00 | | | 3.34 |
| 4 | Purified water | | | 30.00 | | | 20.01 |
| 5 | Ethanol | 95.00 | 97.00 | 65.00 | 59.38 | 64.70 | 43.36 |
| 6 | Propellant AP70 | | | | 37.50 | 33.30 | |
| 7 | Propellant DME | | | | | | 33.30 |
| | TOTAL | 100.00 | 100.00 | 100.00 | 100.00 | 100.00 | 100.01 |

### Method of Preparation and Stability Testing

The L-ascorbic acid (or ascorbyl tetraisopalmitate or ascorbic acid 6-palmitate) was added to the solvent (water and/or ethanol) and stirred until clear. The aerosol base formulations (#1-3) were stored in glass amber vials and the spray formulations (#4-6) were stored in 1 oz plastic coated glass aerosol bottles with an upright valve and dip tube. The aerosol bottles were crimped and charged with propellant to give an aerosol spray.

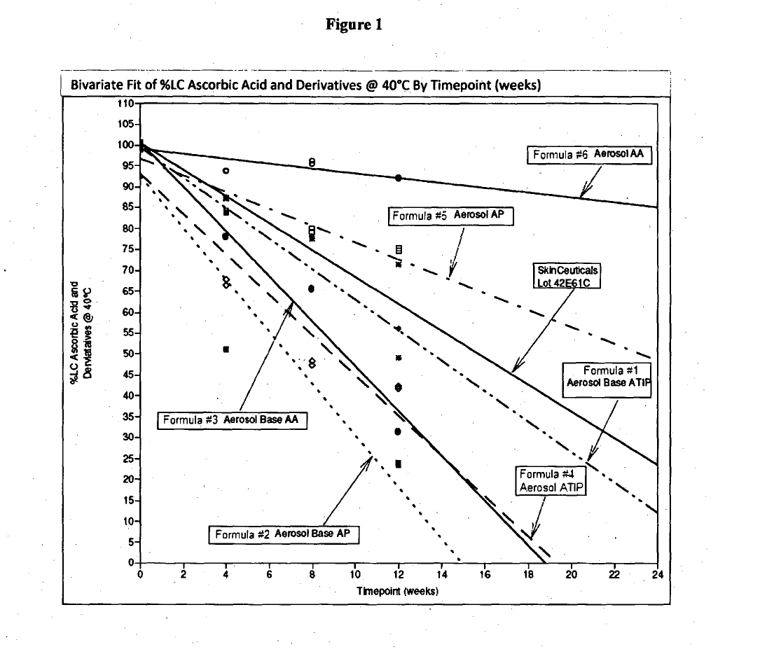

These samples were stored at 40° C. and tested for stability of L-ascorbic acid (or derivative) at t=0, 4, 8 and 12 weeks using HPLC (Waters, Atlantis C₁₈ reverse phase column 250×4.6 mm, 5 μm at 1.2 mL/min with isocratic elution; PDA UV detection; mobile phase: 0.05% H₃PO₄ in water). These analytical conditions were also used in Examples 2 to 7. The stability of the respective samples is illustrated in FIG. 1. It was observed that formulation #6 had the best stability profile over the 12 week period at 40° C.

### Example 2

### Further Stability Testing

The stability of L-ascorbic acid in formulation #6 was unexpectedly high. To confirm the result, the study was repeated. Methyl paraben was also added to formulations #9 and #10 to act as an internal standard, as follows:

TABLE 2

| | | Formulation Number | | | |
|---|---|---|---|---|---|
| | | #7 | #8 | #9 | #10 |
| Item | Ingredients | % w/w | % w/w | % w/w | % w/w |
| 1 | L-Ascorbic acid | 5.00 | 3.34 | 5.00 | 3.34 |
| 2 | Purified water | 30.00 | 20.01 | 30.00 | 20.00 |
| 3 | Ethanol | 65.00 | 43.36 | 64.00 | 42.69 |
| 4 | Methylparaben | | | 1.00 | 0.67 |
| 5 | Propellant DME | | 33.30 | | 33.30 |
| | TOTAL | 100.00 | 100.01 | 100.00 | 100.00 |

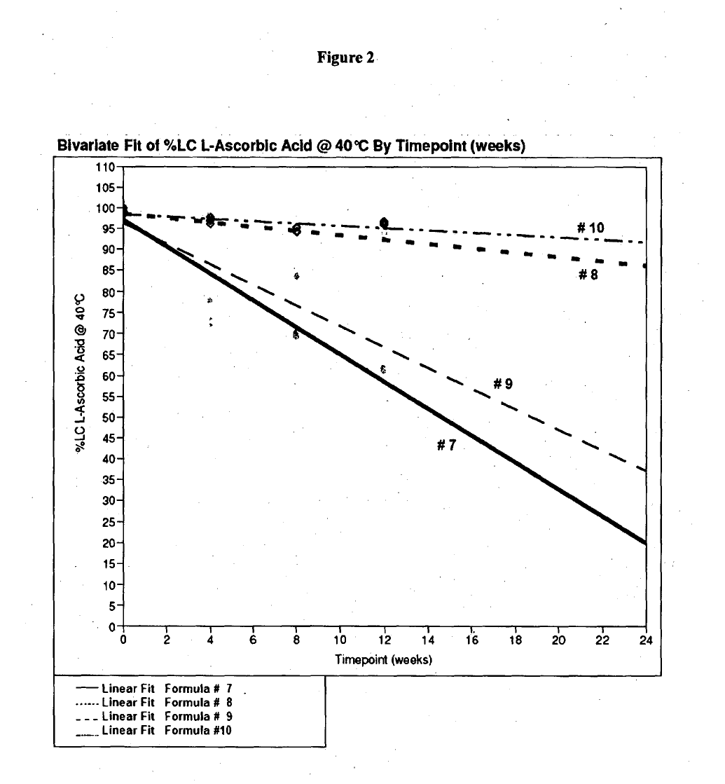

Again, the formulations were prepared by adding the L-ascorbic acid to solvent (water and ethanol), whilst stirring until a clear solution resulted. The aerosol base formulations (#7 and #9) were stored in glass amber vials and the spray formulations (#8 and #10) were stored in polyamide-imide (HOBA PAM 8460) lined aluminium aerosol cans with an upright valve and dip tube. ‘The samples were stored at 5° C. and 40° C. for 12 weeks, and then subjected to analytical testing. The results of the analytical testing are illustrated in FIG. 2. Again, the aerosol spray formulations demonstrated excellent stability, with less than 5% loss after 12 weeks at 40° C.

### Example 3

### Formulation Optimisation

In order to explore whether the choice of propellant influences the stability of L-ascorbic acid, further trials were conducted.

In particular, the trial was designed to investigate whether the improved stability of L-ascorbic acid is specific to the propellant DME, or whether other aerosol propellants could be used to produce similar stability results.

A single bulk batch of aerosol base was prepared (Table 3) using the method described in Examples 1 and 2 and subsequently used to prepare the formulations described in Table 4.

TABLE 3
aerosol base

| Ingredients | % w/w |
|---|---|
| L-Ascorbic acid | 5.00 |
| Purified water | 30.00 |
| Ethanol | 64.90 |
| Methylparaben | 0.10 |
| TOTAL | 100.00 |

TABLE 4

| | | Formula Number | | | | | | |
|---|---|---|---|---|---|---|---|---|
| | | #11 | #12 | #13 | #14 | #15 | #16 | #17 |
| Item | Ingredient | % w/w | % w/w | % w/w | % w/w | % w/w | % w/w | % w/w |
| 1 | Aerosol base | 100.00 | 100.00 | 100.00 | 66.67 | 90.00 | 95.24 | 90.00 |
| 2 | Propellant DME | | | | 33.33 | | | |
| 3 | Propellant AP70 | | | | | 10.00 | | |
| 4 | Propellant N₂ | | | | | | 4.76 | |
| 5 | Propellant 134a | | | | | | | 10.00 |
| | TOTAL | 100.00 | 100.00 | 100.00 | 100.00 | 100.00 | 100.00 | 100.00 |
| | COMMENT | Vacuum | Non- | Vacuum | Vacuum | Vacuum | Vacuum | Vacuum |
| | | crimped | vacuum | crimped/ | crimped | crimped | crimped | crimped |
| | | can only | crimped | N₂ purged | can | can | can | can |
| | | | can | | | | | |
| | | | only | | | | | |

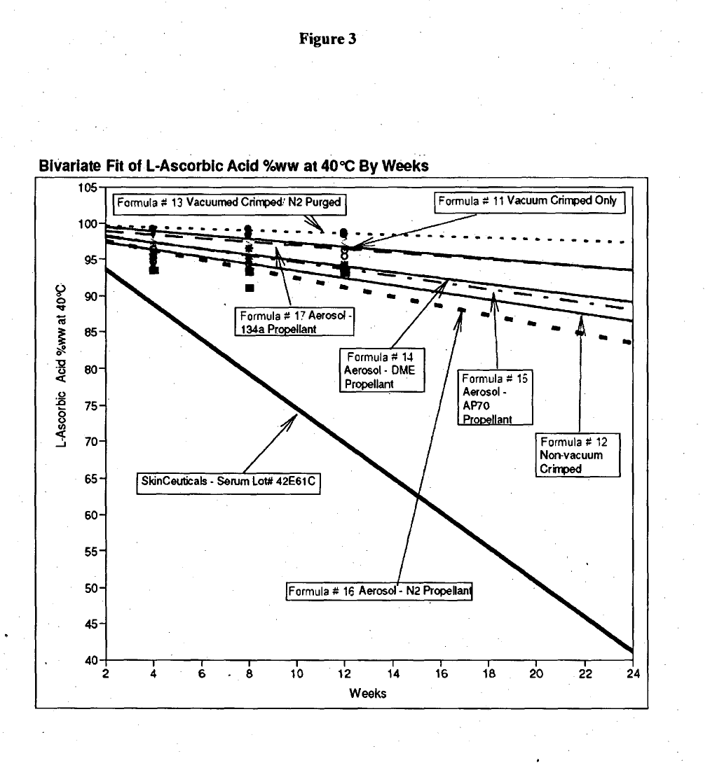

The stability of the respective formulations is illustrated in FIG. 3. All aerosol spray formulations showed less than 15% loss of active after 24 weeks of storage at 40° C. As shown in FIG. 3, the samples prepared in accordance with the present invention demonstrated significantly better stability than the commercial comparator, namely SkinCeuticals™ C E Ferulic serum which contains 15% L-ascorbic acid, ferulic acid and alpha tocopherol.

### Example 4

### L-Ascorbic Acid Aerosol Foam

The following aerosol foams were prepared:

TABLE 5

Formulation Number

| | | #18 | #19 |
|---|---|---|---|
| Item | Ingredients | % w/w | % w/w |
| 1 | Cetyl alcohol | 1.10 | 1.00 |
| 2 | Stearyl alcohol | 0.50 | 1.00 |
| 3 | Polysorbate 60 | 0.40 | 0.40 |
| 4 | Dimethicone | | 0.20 |
| 5 | PEG-75 | | 0.20 |
| 6 | Ethanol | 53.22 | 52.74 |
| 7 | Purified water | 35.48 | 35.16 |
| 8 | L-Ascorbic acid | 5.00 | 5.00 |
| 9 | Propellant AP70 | 4.30 | 4.30 |
| | TOTAL | 100.00 | 100.00 |

Formulation #19 included (i) PEG-75 as a humectant and (ii) dimethicone (Dow Corning 200 fluid, 0.65 cst) as a skin conditioning agent.

### Method of Preparation

The aerosol foams were prepared as follows:
1. Combine together items 1 to 6. Warm gently until clear. Put aside until required (ethanol phase).
2. In a separate beaker combine items 7 and 8. Stir until clear (water phase).
3. Separately fill the ethanol phase and water phase into an aerosol can.
4. Crimp and gas (item 9).

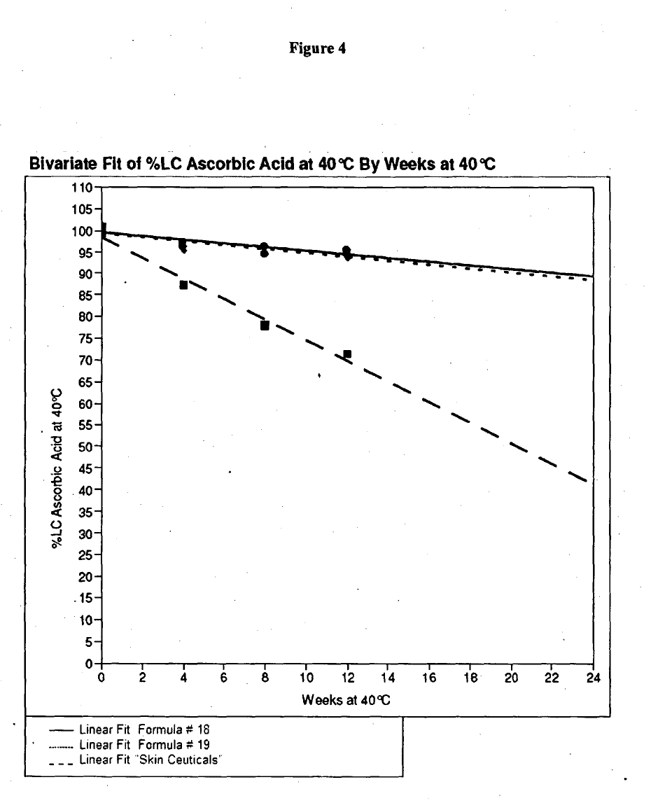

The foams were stored at 40° C. and subjected to stability analysis. The results of the analysis are illustrated in FIG. 4. L-Ascorbic acid was particularly stable in the foam vehicle. Moreover, the addition of PEG-75 and dimethicone did not impact the stability of the L-ascorbic acid. As shown in FIG. 4, the foam samples displayed superior stability relative to the commercial comparator (SkinCeuticals™ C E Ferulic serum).

### Example 5

### L-Ascorbic Acid Plus Green Tea Aerosol Foam

TABLE 6

| | | Formulation number |
|---|---|---|
| Item | | #20 |
| no. | Ingredient | % w/w |
| 1 | Cetyl alcohol | 1.10 |
| 2 | Stearyl alcohol | 0.50 |
| 3 | Polysorbate 60 | 0.40 |
| 4 | Ethanol | 52.56 |
| 5 | Purified water | 35.04 |
| 6 | Disodium EDTA | 0.10 |
| 7 | L-Ascorbic acid | 5.00 |
| 8 | Camellia sinensis leaf extract | 1.00 |
| 9 | Propellent AP70 | 4.30 |
| | TOTAL | 100.00 |

### Method of Preparation

The aerosol foam was prepared as follows:
1. Combine together items 1 to 4. Warm gently until clear. Put aside until required (ethanol phase).
2. In a separate vessel, combine items 5 and 6. Stir until clear.
3. Whilst stirring, add item 7 and stir until clear.
4. Whilst stirring, add item 8 and stir until clear (water phase).
5. Separately fill the ethanol phase and water phase into an aerosol container.
6. Crimp and gas (item 9).

### Example 6

### Further L-Ascorbic Acid Foams

TABLE 7

| | | Formulation number | | | |
|---|---|---|---|---|---|
| Item | | #21 | #22 | #23 | #24 |
| no. | Ingredients | w/w % | w/w % | w/w % | w/w % |
| 1 | Ethanol | 28.500 | 38.000 | 47.880 | 50.220 |
| 2 | Cetyl Alcohol | 0.665 | 0.665 | 1.045 | 1.100 |
| 3 | Stearyl Alcohol | 0.285 | 0.285 | 0.475 | 0.500 |
| 4 | Steareth-20 | 0.475 | 0.475 | 0.000 | 0.000 |
| 5 | Polysorbate 60 | 0.000 | 0.000 | 0.380 | 0.400 |
| 6 | Purified Water | 49.875 | 40.375 | 30.020 | 33.480 |
| 7 | PEG-75 | 1.900 | 1.900 | 1.900 | 0.000 |
| 8 | L-Ascorbic Acid | 11.400 | 11.400 | 11.400 | 10.000 |
| 9 | Water, glycerin and | 0.950 | 0.950 | 0.950 | 0.000 |
| | Piptadenia Colubrina | | | | |
| | Peel extract¹ | | | | |
| 10 | Water, glycerin and | 0.950 | 0.950 | 0.950 | 0.000 |
| | Physalis Angulata extract² | | | | |
| 11 | AP70 hydrocarbon | 5.000 | 5.000 | 5.000 | 4.300 |
| | propellant | | | | |
| | TOTAL | 100.000 | 100.000 | 100.000 | 100.000 |

¹Aquasense 3R (Chemyunion, San Paulo, Brazil)
²Ecophysalis (Chemyunion, San Paulo, Brazil)

### Method of Preparation (Formulations #21 and #22)

1. Combine together items 1 to 3. Stir gently until clear. Put aside until required (ethanol phase).
2. In a separate beaker, combine items 4 and 6. Stir until clear.
3. Continue stirring and add item 7. Stir until clear.
4. Continue stirring and add item 8. Stir until clear.
5. Continue stirring and add item 9. Stir until clear.
6. Continue stirring and add item 10. Stir until clear (water phase).
7. Separately fill the ethanol phase and the water phase into an aerosol can.
8. Crimp and gas (item 11).

### Method of Preparation (Formulation #23)

1. Combine items 1 (60% of total ethanol), 2, 3 and 5. Stir gently until clear. Put aside until required (ethanol phase).
2. In a separate beaker, combine items 6 and 7. Stir until clear.
3. Continue stirring and add item 8. Stir until clear.
4. Continue stirring and add item 9. Stir until clear.
5. Continue stirring and add item 10. Stir until clear.
6. Continue stirring and add remaining item 1 (40% of total ethanol). Stir until clear (water phase).
7. Separately fill the ethanol phase and the water phase into an aerosol can.
8. Crimp and gas (item 11).

### Method of Preparation (Formulation #24)

1. Combine items items 1, 2, 3 and 5. Stir gently until clear. Put aside until required (ethanol phase).
2. In a separate beaker, combine items 6 and 8. Stir until clear (water phase).
3. Separately fill the ethanol phase and the water phase into an aerosol can.
4. Crimp and gas (item 11).

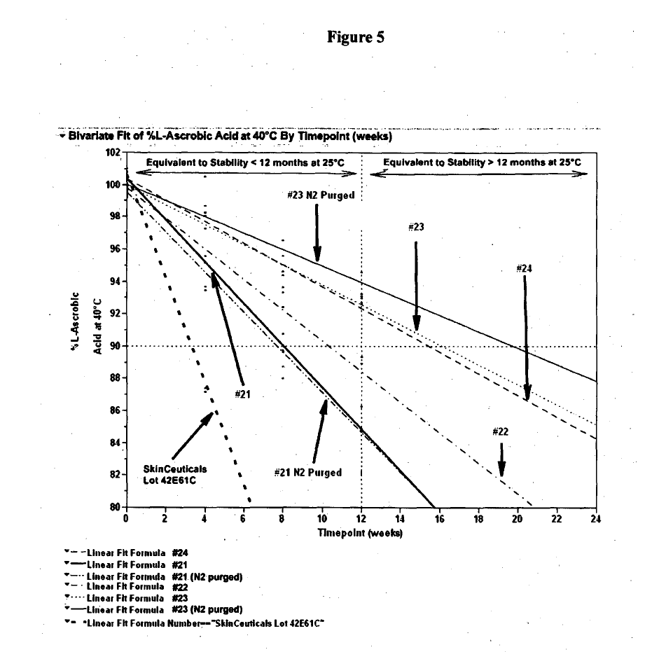

The foam formulations were stored at 40° C. and subjected to stability analysis. The results of the analysis are shown in FIG. 5. A relationship between ethanol content and stability of L-ascorbic acid was observed. That is, increased ethanol content affords improved stability of L-ascorbic acid.

### Example 7

### L-Ascorbic Acid Plus Coffeeberry™ Extract Aerosol Foam

TABLE 8

| | | Formulation number |
|---|---|---|
| Item | | #25 |
| no. | Ingredients | % w/w |
| 1 | Cetyl alcohol | 0.665 |
| 2 | Stearyl alcohol | 0.285 |
| 3 | Steareth-20 | 0.475 |
| 4 | Ethanol | 28.500 |
| 5 | Purified water | 48.925 |
| 6 | PEG-75 | 1.900 |
| 7 | L-Ascorbic acid | 11.400 |
| 8 | Coffee Arabica fruit extract¹ | 0.950 |
| 9 | Water, glycerin and | 0.950 |
| | Piptadenia Colubrina Peel extract² | |
| 10 | Water, glycerin and | 0.950 |
| | Physalis Angulata extract³ | |
| 11 | Propellant AP 70 | 5.000 |
| | TOTAL | 100.000 |

¹Coffeeberry ™ extract (FutureCeuticals, IL, USA)
²Aquasense 3R (Chemyunion, San Paulo, Brazil)
³Ecophysalis (Chemyunion, San Paulo, Brazil)

### Method of Preparation

1. Combine together items 1 to 4. Stir gently until clear. Put aside until required (ethanol phase).
2. In a separate beaker combine items 5 and 6. Stir until clear.
3. Continue stirring and add item 7. Stir until clear.
4. Continue stirring and add item 8. Stir until clear.
5. Continue stirring and add item 9. Stir until clear.
6. Continue stirring and add item 10. Stir until clear (water phase).
7. Separately fill the ethanol phase and the water phase into an aerosol can.
8. Crimp and gas the aerosol can with propellant (item 11).

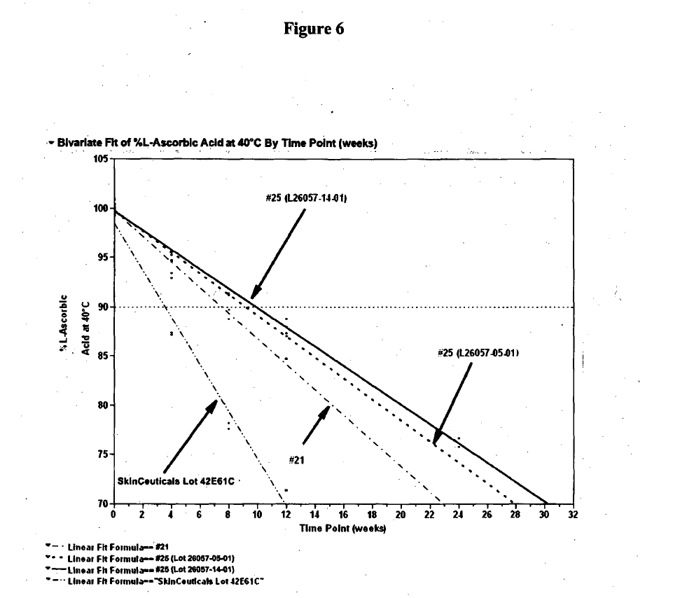

The formulation was stored at 5° C., 25° C. and 40° C. and subjected to stability analysis. The results of the analysis following storage at 40° C. are shown in FIG. 6. The stability of L-ascorbic acid in Formulation #25 (L-ascorbic acid plus Coffeeberry™ extract) was superior to comparative Formulation #21 (L-ascorbic acid only). This suggests that Coffeeberry™ extract has a stabilizing effect on L-ascorbic acid.

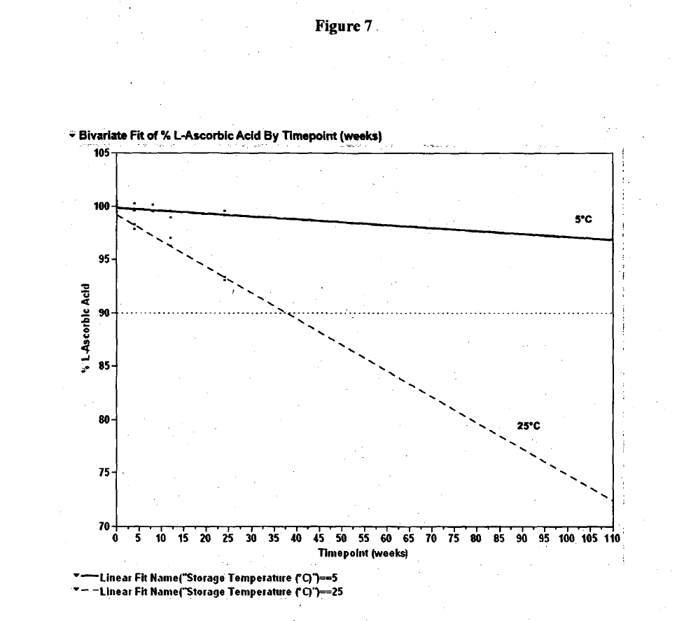

FIG. 7 illustrates the stability of L-ascorbic acid in Formulation #25 at 5° C. and 25° C. (up to 24 weeks of storage). On the basis of the stability testing, it was predicted that less than 10% L-ascorbic acid will be lost after approximately 30 weeks at 25° C. and less than 5% will be lost after approximately 2 years at 5° C.

The following formulations were also prepared using a similar method:

TABLE 9

| | | Formulation number | |
|---|---|---|---|
| Item | | #26 | #27 |
| no. | Ingredients | % w/w | % w/w |
| 1 | Cetyl alcohol | 0.943 | 0.665 |
| 2 | Stearyl alcohol | 0.472 | 0.285 |
| 3 | Steareth-20 | 0.943 | 0.475 |
| 4 | Ethanol | 28.300 | 28.500 |
| 5 | Purified water | 48.400 | 49.685 |
| 6 | PEG-75 | 0.943 | 0.950 |
| 7 | L-Ascorbic acid | 11.321 | 11.400 |
| 8 | Coffee Arabica fruit extract¹ | 0.943 | 0.950 |
| 9 | Water, glycerin and | 0.943 | 0.950 |
| | Physalis Angulata extract² | | |
| 10 | Water, glycerin and | 0.943 | 0.950 |
| | Piptadenia Colubrina Peel extract³ | | |
| 11 | Caprylic/capric triglyceride, | 0.189 | 0.190 |
| | Citris Tangerina Peel extract⁴ | | |
| 12 | Propellant AP 70 | 5.660 | 5.000 |
| | TOTAL | 100.000 | 100.000 |

¹Coffeeberry ™ extract (FutureCeuticals, IL, USA)
²Ecophysalis (Chemyunion, San Paulo, Brazil)
³Aquasense 3R (Chemyunion, San Paulo, Brazil)
⁴Sensique Tangerine (Tri-K, NJ, USA)

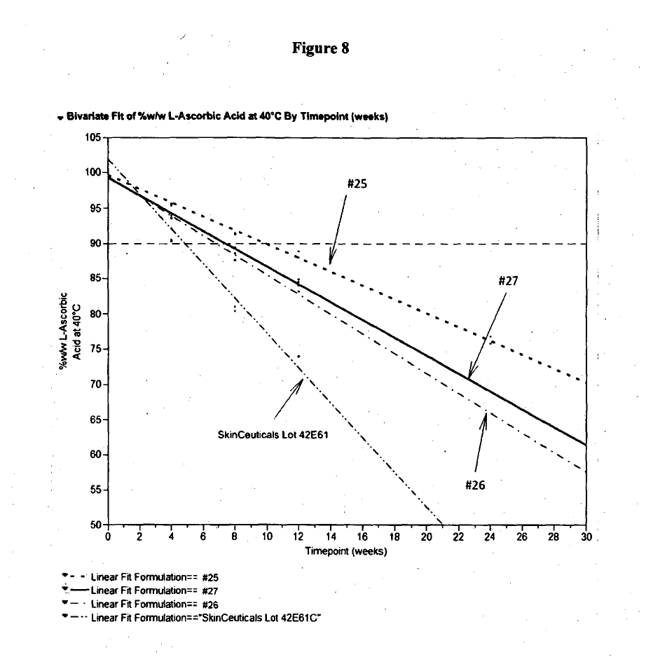

FIG. 8 illustrates the stability of L-ascorbic acid in Formulation #26 and #27 (compared with Formulation #25) following storage at 40° C.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilise the present invention to its fullest extent. Therefore, the examples herein are to be construed as merely illustrative and not a limitation of the scope of the present invention in any way. The embodiments of the invention in which an exclusive property or privilege is claimed are defined as follows.

1. A topical cosmetic composition in an aerosol container comprising
(a) L-ascorbic acid or a pharmaceutically acceptable salt or ester thereof,
(b) water present in an amount from about 10% to about 80% by weight,
(c) a lower (C₁-C₆ alkyl) alcohol,
(d) optionally a dermatologically acceptable excipient, and
(e) an aerosol propellant.

2. The composition according to claim 1, where the L-ascorbic acid or a pharmaceutically acceptable salt or ester thereof is L-ascorbic acid.

3. The composition according to claim 1, wherein the water is present in an amount from about 20% to about 60% by weight.

4. The composition according to claim 1, wherein the lower alcohol is selected from the group consisting of ethanol, propanol, isopropanol, n-butyl alcohol and t-butyl alcohol, and mixtures thereof.

5. The composition according claim 4, wherein the lower alcohol is ethanol.

6. The composition according to claim 1, wherein the lower alcohol is present in an amount from about 20% to about 60% by weight.

7. The composition according to claim 1, wherein the aerosol propellant is selected from the group consisting of a hydrocarbon, dimethyl ether, a chlorofluorocarbon, a hydrofluorocarbon, and mixtures thereof.

8. The composition according to claim 7, wherein the aerosol propellant is a hydrocarbon propellant.

9. The composition according to claim 1, comprising a dermatologically acceptable excipient selected from the group consisting of a preservative, a second antioxidant, a chelating agent, a humectant and a pH adjusting agent, and mixtures thereof.

10. The composition according to claim 1, wherein the composition forms an aerosol spray after release from said container.

11. The composition according to claim 10, wherein the aerosol spray comprises an aerosol propellant in an amount from about 25% to about 40% by weight.

12. The composition according to claim 1, wherein the composition forms an aerosol foam after release from said container.

13. The composition according to claim 12, wherein the aerosol foam comprises an aerosol propellant in an amount from about 3% to about 10% by weight.

14. The composition according to claim 13, further comprising a surfactant and/or a wax.

15. The composition according to claim 14, wherein the surfactant is selected from the group consisting of an ethoxylated fatty alcohol ether, a PEG derivative, an ethoxylated fatty acid, a propylene glycol ester, a glycerol ester or derivative, a polymeric ether and a sorbitan derivative, and mixtures thereof.

16. The composition according to claim 15, wherein the surfactant comprises an ethoxylated fatty alcohol ether.

17. The composition according to claim 14, wherein the wax is a fatty alcohol.

18. The composition according to claim 17, wherein the fatty alcohol is a mixture of stearyl alcohol and cetyl alcohol.

19. The composition according to claim 1, wherein the composition further comprises a nutritional agent.

20. A topical cosmetic composition in an aerosol container comprising:
(a) L-ascorbic acid or a pharmaceutically acceptable salt or ester thereof,
(b) water in an amount from about 40% to about 60% by weight,
(c) a lower alcohol in an amount from about 20% to about 40% by weight,
(d) a wax,
(e) a surfactant,
(f) optionally one or more dermatologically acceptable excipients selected from the group consisting of a preservative, a second anti-oxidant, a chelating agent, a humectant and a pH adjusting agent, and
(g) an aerosol propellant in an amount from about 3% to about 10% by weight, wherein the composition forms an aerosol foam after release from said container.

21-22. (canceled)

23. A method of treating a condition of the skin in a mammal in need thereof comprising administering to said mammal a topical composition according to claim 1.

24. The method according to claim 23 wherein the condition of the skin is aging of the skin.
